(19) United States
(12) Patent Application Publication
(10) Pub. No.: US 2019/0365571 A1
(43) Pub. Date: Dec. 5, 2019

(54) A DRESSING SYSTEM

(71) Applicants: FLEMING MEDICAL LTD., Limerick (IE); UNIVERSITY COLLEGE CORK - NATIONAL UNIVERSITY OF IRELAND, Cork (IE)

(72) Inventors: Conor O'MAHONY, Cork (IE); Brendan O'FLYNN, Cork (IE); Paul GALVIN, Cork (IE); Mark FLEMING, Limerick (IE)

(21) Appl. No.: 16/472,786

(22) PCT Filed: Dec. 22, 2017
(86) PCT No.: PCT/EP2017/084424

§ 371 Date: Jun. 21, 2019

(30) Foreign Application Priority Data
Dec. 22, 2016 (EP) 16206492.7

(51) Int. Cl. A61F 13/00; A61B 5/00

(52) U.S. Cl. CPC A61F 13/00085; A61F 13/00055; G16H 40/63; A61B 5/445; A61B 5/6833

(57) ABSTRACT

The invention provides a dressing system for a wound comprising an absorbent pad; at least one sensor for detecting wound or dressing data, wherein the at least one sensor is a moisture sensor; a flexible electronic circuit communicable with the sensor; a backing film adapted to cooperate with the absorbent pad, wherein the electronic circuit and the at least one sensor is printed directly on the backing film; and a communications module adapted to communicate the wound data from the electronic circuit to a user or clinician. The invention provides a simple to use disposable dressing wound system that is effective in providing dressing wound data that provides an indicator of the status of the wound as well as information that the dressing wound system needs to be replaced.

### INTRODUCTION

This invention relates to a wound dressing system having one or more sensors for monitoring and treating a chronic wound and to a method for monitoring a chronic wound, and to monitoring the condition of the dressing itself.

### BACKGROUND

A major requirement for effective wound care management is the need to monitor and change wound dressings to provide optimal conditions for tissue healing. Current methods of wound monitoring and care require manual and/or subjective analysis of a range of parameters such as wound temperature and wound dressing saturation/age which usually involves disturbing the patient and/or removing the dressing for inspection. For example, it is recognized that wound temperature is a quantitative measurement that has the potential to assist in assessing and diagnosing chronic deep wound and surrounding skin infection. Indeed, studies have shown that incorporating quantitative skin temperature measurement into routine wound assessment provides a timely and reliable method for a clinician to quantify the heat associated with deep wound and surrounding skin infection (e.g. elevated local temperatures above 37° C. are an indication of wound infection) and to monitor ongoing wound status.

Similarly, it is known that, as healing occurs, the amount of exudate produced usually decreases. The volume of exudate is related to the surface area of the wound and large wounds often produce higher volumes of exudate. However, although a moist wound environment is necessary for optimal wound healing, over- or under-production of exudate may adversely affect healing. Any factor that increases capillary leakage or predisposes a patient to the development of tissue oedema (e.g. inflammation, bacterial contamination or limb dependency) may boost exudate production while low exudate production may indicate a systemic problem (e.g. dehydration, hypovolemic shock and microangiopathy) or may be a feature of ischaemic ulcers. Accordingly, it is essential to accurately determine and evaluate the factors contributing to the production of too much or too little exudate.

However, currently, measurement and assessment of the aforementioned and other wound parameters must be performed manually using subjective visual inspections and/or equipment such as thermometers or moisture meters resulting in suboptimal dressing changing frequency, clinical timewasting, patient discomfort, increased dressing cost and increased wound healing time.

Numerous patent publications exist in the art, for example US2016/015962 concerned with photo-stimulation and sensing of wound response. A reusable electronic device is constructed from a multiple number of discrete layers separate to the outermost backing films, and the device is intended to be separable and partially reusable after use. US2016/015962 describes a flexible patch that uses LEDs to accelerate the wound healing process, sensors to provide closed-loop feedback on that healing process, and wireless communications to relay information to a clinician. It is an active device that is complex and expensive to manufacture. Other patent publications in the art include WO2016/166731 and US2015/200538.

It is therefore an object to provide an improved dressing system and method to overcome the above mentioned problems.

### SUMMARY OF THE INVENTION

According to the invention there is provided, as set out in the appended claims, a dressing system for a wound comprising:
an absorbent pad;
at least one sensor for detecting wound or dressing data, wherein the
at least one sensor is a moisture sensor;
a flexible electronic circuit communicable with the sensor;
a backing film adapted to cooperate with the absorbent pad, wherein the electronic circuit and the at least one sensor is printed directly on the backing film; and
a communications module adapted to communicate the wound data from the electronic circuit to a user or clinician.

The invention provides a one-time use disposable and integrated dressing wound system. The sensors and electronic circuity are printed on to a suitable single substrate layer onto which components such as sensors, microcontrollers and antennae are also printed or mounted using standard electronic packaging technologies. This substrate may comprise backing layers, wicking layers and/or absorption layers which already form part of a standard dressing, thereby negating the need for any additional layers other than those which are required by the prior art. Significant savings in manufacturing and assembly costs may be achieved using this approach, which furthermore results in a dressing that low-profile, comfortable and is more flexible to those proposed in the art. This substrate layer with the printed or mounted components can then cooperate with the absorption layer. The invention provides a simple to use disposable dressing wound system that is effective in providing dressing wound data that provides an indicator of the status of the wound as well as information that the dressing wound system needs to be replaced.

The invention provides a means of monitoring the condition of the wound, and assessing, for example, whether the wound is heavily or lightly exuding, whether infection is present, and what the status of the healing process is.

Furthermore, the invention provides a means of monitoring the condition of the dressing itself, and detecting, for example, the volume of exudate absorbed by the dressing, the remaining capacity of the dressing to absorb further exudate, and when the dressing may require changing. It will be appreciated that monitoring the conditions of the dressing itself provide data to infer the condition of the actual wound.

Advantageously, the communications module comprises a wireless communications module.

Preferably, the sensor comprises an internal sensor in the absorbent pad. More preferably, the internal sensor comprises a pH sensor. Alternatively, the internal sensor comprises a moisture sensor. Preferably, the moisture sensor comprises an impedance sensor, capacitance sensor, resistance sensor or strain sensor.

Alternatively, the internal sensor comprises a force sensor. Preferably, the force sensor comprises an electrical force sensor.

Alternatively, the internal sensor comprises a bacterial sensor.

Suitably, the sensor comprises an internal sensor integrated within the absorbent pad. Preferably, the internal sensor comprises a pH sensor.

Alternatively, the dressing system comprises a sensor on the dressing border. Preferably, on the peripheral region of the wound comprising a temperature sensor.

Alternatively, the sensor comprises an inertial sensor or accelerometer for monitoring patient orientation and activity. The inertial sensor or accelerometer is adapted to provide information on the movement and orientation of a patient.

Optimally, the sensor comprises a sensor network. Preferably, the sensor network comprises sensor nodes.

Preferably, the dressing system comprises a power source for powering the electronic circuit. More preferably, the power source comprises a battery. Advantageously, the battery may be of flexible construction and/or suitable for incineration. For example the battery can be an organic battery suitable for safe disposable or incineration.

In one embodiment, the circuit is manufactured on a printed circuit board (PCB). Preferably, the PCB is made from a flexible material. The PCB may be detachable form the dressing system.

Suitably, the dressing further comprises a backing film on the absorbent pad. Preferably, the backing film comprises a polyurethane backing film.

Advantageously, the electronic circuit is printed directly on the backing film and associated microelectronic components mounted thereupon.

Alternatively, the electronic circuit is printed on the absorbent pad. Preferably, the electronic circuit is printed on a single side of the absorbent pad. More preferably, some or all parts of the electronic circuit are printed on two sides of the absorbent pad. In a further embodiment, two or more such printed absorbent pads are stacked to form a multilevel circuit.

In one embodiment of the invention, the absorbent pad comprises a secondary absorption pad in the dressing. Preferably, the secondary absorption pad comprises a foam. The secondary absorption pad may comprise a plurality of stacked absorption pads, wherein the electronic circuit is printed on the plurality of absorption pads to form a multilevel circuit.

Suitably, the electronic circuit comprises a processor for processing data from the sensors and memory for storing the wound data.

Preferably, the dressing system further comprises a base station for receiving the communicated wound data from the communications module. More preferably, the base station comprises a sink node.

The invention also extends to a method for monitoring a wound comprising:
applying a dressing system having at least one sensor at the peripheral region of the wound;
detecting wound or dressing data at the sensor;
communicating the detected wound data via an electronic circuit in the dressing system to a base station, and
displaying the data at the base station or associated handheld device.

Preferably, the detected wound data is wirelessly communicated to the base station.

Preferably, the signal strength and associated attenuation of the communicated radio signal is used to monitor patient position orientation and location. The wound data may comprise one or more of: orientation data, activity data or movement data.

Preferably, the wound data comprises pH data. Alternatively, the wound data comprises temperature data. Alternatively, the wound data comprises moisture data. Alternatively, the wound data comprises force data. Alternatively, the wound data comprises bacterial data. Alternatively, the wound data comprises patient orientation and movement data.

Preferably, the method further comprises the step of processing the data prior to displaying the data at the base station.

More preferably, a biostatistical analysis is performed on the processed data to output a graphically represented wound healing trend.

The absorbent pad may be provided with a cavity to receive the internal sensor.

More preferably, an analysis is performed on the processed data to output a wound healing trend.

The method may further comprise displaying the wound healing trend as a graphical representation.

The method may further comprise monitoring the strength of the data received at the base station.

The method may further comprise determining one or more of the position, orientation and location of a patient associated with the wound data based on the strength of the data received at the base station.

The invention therefore results in a smart or intelligent wound dressing for the intelligent monitoring of wound health and dressing condition. The dressing is low-profile, easily embedded within conventional dressing architectures and contains sensors and associated electronics for the detection and recording of conditions such as dressing moisture/saturation levels and/or wound temperature to provide clinicians with a better understanding of the specific wound healing process of individual patients.

The dressing of the invention is a laminated or multi-layer dressing adapted to rapidly absorb exudates and interstitial fluids and optimize conditions for healing at the wound-dressing interface. A primary wicking or absorbent pad provides a rapid capillary action response to quickly distribute absorbed exudate throughout the dressing and create a sustained movement of fluid away from wound beds. In one form, the dressing covers a wound and an area of skin surrounding the wound and has at least one sensor on an external layer of a secondary absorption pad and three or more sensors on the internal section of the secondary absorption pad with the flexible electronic circuit printed on the secondary absorption pad.

In another form, the electronic circuit is printed directly onto the internal face of the backing film and the sensors can be located underneath laser-cut cavities in the secondary absorption pads for low profile sensor embedding. Accordingly, specific sensors can be integrated into the dressing without requiring relatively large secondary absorption pads resulting in improved sensor readings. The secondary absorption pad can then be directly fabricated onto the backing film.

The dressing of the invention facilitates the monitoring of individual and multiple sensor readings of a wound in real-time and/or the provision of time specific updates. The data generated from the dressing provides a constant and/or time specific update to an app and/or software available to a clinician. Each sensor reading provides a specific reading that is easily interpreted for ease of use and understanding.

The pH sensor facilitates the measurement of exudate pH to reflect the condition of a wound bed and aid in monitoring and determining the wound's response to treatment. The moisture sensor quantitatively monitors the wound exudate and is calibrated for measuring high and low absorption capacity to indicate when the primary absorbent pad and/or the secondary absorption pad is dry or wet respectively. The pressure sensor reading provides clinicians with key evidence describing pressure applied on wounds during critical healing stages. The inertial sensor provides information on patient activity levels and orientation. Analysis of the received radio signal strength further provides information on patient orientation.

Where the sensors of the dressing are deployed as a sensor network in the form of sensor nodes densely deployed within the dressing, otherwise difficult-to-access wounds can be monitored with ease.

The dressing of the invention provides a sealing arrangement preventing any bodily fluids or other material that may cause infection and/or impact sensor readings from reaching a wound. The waterproof sealing arrangement also enables the patient to wash and shower without damaging or obstructing the dressing. This is particularly beneficial when the dressing is employed on long term wounds.

The integrated printed electronic circuit with sensors results in a dressing having great flexibility in design and functionality with the printed electronic circuit allowing for dynamic and repetitive dressing flexing. A two or double-sided, or multilevel, flexible printed circuit offers the same level of dynamic, repetitive flexing as the single-sided printed electronic circuit but with a greater range of application due to the ability to carry more complex circuit layouts. Such an arrangement is extremely advantageous due to greater circuit design parameters and reduced assembly costs resulting from minimized interconnect errors. Reduced packaging dimensions are also advantageous in such a design.

Data generated from the dressing is used for the prediction of wound healing to help clinicians adopt a more specific management strategy, at the right time, to achieve healing. The algorithm employed to process the data is based on recurrent trends within the data generated to predict patient healing and or wound implications e.g. slow healing time of specific wound types. The use of the sensed parameters (e.g. pH, exudate and temperature) in combination provides essential information that improves future patient care.

The data harvested from the sensors of the dressing can be used to construct a model describing the sensed parameters so that clinician queries can be answered using the model instead of the actual sensed data. Two communication models can be employed—one residing at the sink and the other at the sensors—so that the model at the sink can be used to answer queries without requiring any communication thus reducing energy consumption.

The data transferred to a downloaded app on a handheld device can be used to provide clinicians with continuous data for monitoring and analysing wounds with the app enabling real-time bi-directional communication between patient and clinician.

### BRIEF DESCRIPTION OF THE DRAWINGS

The invention will now be described, by way of example only, with reference to the accompanying drawings in which:

FIG. 1 is an exploded perspective view from above and one side of an intelligent wound dressing system of the invention for monitoring and treating a wound with the layers of the dressing separated to reveal the embedded sensors and flexible integrated circuitry within the dressing with the direction of movement of exudate from a wound through the wicking primary absorbent pad of the dressing indicated by arrows;

FIG. 2 is a plan view from below of the intelligent dressing system of FIG. 1 with the absorbent polymer and absorbent pad layers removed to reveal the embedded sensors and flexible integrated circuitry of the dressing;

FIG. 3 is a side elevation of the dressing system of FIG. 2; and

FIG. 4 is an exploded perspective view from above and one side of an intelligent disposable wound dressing system of the invention for applying the sensor and circuitry directly onto a wound.

### DETAILED DESCRIPTION OF THE INVENTION

As shown in the drawings, an intelligent laminate dressing system for monitoring and treating a wound is generally indicated by the reference numeral 1 and is made up of a primary wicking absorbent pad 2 for absorbing wound exudate, an optional secondary polymeric foam absorption pad 3 having a flexible wound monitoring electronic circuit 4 printed thereon and an adhesive backing film 5 attached to the secondary absorption pad 3 defining a peripheral adhesive side border 6 for adhering the dressing 1 to a patient. The dressing 1 is further provided with a removable release film 7 on the primary absorbent pad 2 for protecting the dressing 1 prior to use.

The primary absorbent pad 2 is made up of a substantially square lower wound face 8 for contacting a wound having four side walls 9,10,11,12 upstanding therefrom and an upper face 13 disposed towards the secondary absorption pad 3. The wound face 8 is provided with an absorbent polymer layer 14 which can be stick or non-stick as required for directly contacting a wound while the adhesive side border 6 of the backing film 5 is provided with an adhesive 15 for adhering the dressing 1 to the patient around the wound.

The optional secondary absorption pad 3 is shaped and dimensioned like the primary absorbent pad 2 and is provided with an outer lower absorbent face 16 on which the electronic circuit 4 is printed disposed towards the primary absorbent pad 2 and an outer upper backing film face 17 disposed towards the backing film 5. It will be appreciated that in one embodiment the electronic circuit can be printed on the adhesive film backing 5 with no need for the secondary pad 3. It will be appreciated in a simple embodiment of the invention there is a single absorbent pad 2, 3 with the electronic circuit and sensors printed thereon. Effectively the electronic circuit, power and the sensors are a printed Integrated Circuit (IC).

The dressing system 1 is provided with embedded physiological and/or biological wound sensors 18 communicable with the electronic circuit 4. In the present embodiment, the sensors 18 are made up of internal sensors 19 contained within the secondary absorption pad 3, an external sensor 20 on the printed circuit face 16 of the secondary absorption pad 3 and a peripheral sensor 21 positioned beyond the secondary absorption pad 3 (and the primary absorbent pad 2) at the adhesive side border 6 but communicable with the electronic circuit 4.

The internal sensors 19 include a pH sensor 22 for measuring the pH of exudate.

The pH sensor 22 is located within the secondary absorption pad 3 and communicable with the printed electronic circuit 4. The pH sensor 22 can also be integrated within the electronic circuit 4. By monitoring the pH of exudate, the pH sensor 22 provides information on the condition of the wound bed and aids in determining the wound's response to treatment. The pH sensor 22 has a measuring range of 0.00-14.00. The pH sensor can be calibrated to be accurate within ±0.01 of the pH measured as a resolution value of 0.01 is required for accurate determination of exudate pH. The pH sensor 22 has a power consumption of 5 mW-10 mW.

The pH sensor 22 can also be employed as an external sensor 20 if desired e.g. on the surface of the primary absorbent pad 2.

The optional secondary absorption pad 3 is provided with an additional internal sensor 19 in the form of an exudate moisture detecting sensor 23. In addition, the electrodes that comprise this sensor may be printed directly on the absorption pad or backing layer. In one embodiment, the moisture sensor is an electrical impedance sensor that is calibrated to measure high and low impedance—a high impedance reading indicates that the primary absorbent pad 2 and secondary absorption pad 3 are dry (i.e. low exudate levels) while a low impedance indicates that the primary absorbent pad 2 and secondary absorption pad 3 are wet (i.e. high exudate levels).

In a further embodiment, the sensor is a capacitive moisture sensor, the capacitance of which varies from a low value for a dry pad (e.g. 0.2 pF) to a high value for a wet pad (e.g. 200 pF).

In a further embodiment, the sensor is a resistive moisture sensor, the resistance of which varies from a high value for a dry pad (e.g. 3 MOhms) to a low value for a wet pad (e.g. 1 kOhms).

As indicated above, it is critical for effective healing to know the quantity of exudate that is being produced by a wound as, although a moist wound environment is necessary for optimal wound healing, over- or under-production of exudate can adversely affect healing. The moisture readings also provide optical, audible, or electronic indicators as to the correct time to change the dressing system 1 by generating a signal via the electronic circuit 4. A suitable moisture detecting impedance sensor 23 is a pair of small, silver chloride electrodes employing a low current with an optimal moisture level for wound healing being defined as an intermediate impedance range between the high and low values described above i.e. a desired moist condition can be defined as a range of impedance located between a high and a low value.

A suitable moisture detecting capacitance sensor 23 consists of a pair of planar electrodes, typically arranged in an interdigitated pattern and covered with a material to prevent an electrical short circuit. Since the relative dielectric permittivity of the pores in the foam will vary from approximately 1 (when dry and air filled) to approximately 80 (when exudate saturated), the capacitance between the electrodes will vary accordingly, i.e. a desired moist condition can be defined as a range of capacitance located between a high and a low value.

A suitable moisture detecting resistance sensor 23 consists of a pair of planar electrodes, typically arranged in an interdigitated pattern and in direct contact with the fluid exudate. Since exudate that is in direct contact with both electrodes will provide a low resistance patch between those electrodes, the resistance between the electrodes will vary according to the amount of exudate present. A desired moist condition can be defined as a range of resistance located between a high and a low resistance value. It will be appreciated that the sensors can be configures to measure wound data directly or dressing wound data depending on the application required.

An internal force sensor 24 is also provided at the secondary absorption pad 3 for providing an indication of the force applied to a wound by a patient. In one embodiment, the force sensor is made up of an elongated, flexible support strip (not shown) on the secondary absorption pad 3. The support strip is made up of a flat pressure sensitive portion having electrical properties which vary with force applied perpendicular to the plane defined by the support strip. The support strip is communicable with the electronic circuit 4 to provide readouts of the detected force. The force sensor 24 provides an indication of the force being applied to pressure specific wounds whilst the dressing system 1 simultaneously protects the wound. The force sensor 24 reading provides clinicians with key evidence describing pressure applied on wounds during critical healing stages.

The internal sensor 19 can also be a bacterial sensor.

An inertial sensor, such as a three-axis accelerometer, is also provided at the secondary absorption pad 3 for providing an indication of the motion of the patient. The sensor can provide information about the orientation and activity levels of the patient. It may be used to monitor the frequency of changing the patient's position in bed, patient gait and ambulatory information, fall detection, etc.

The dressing system 1 is further provided with a peripheral sensor 21 in the form of a temperature sensor 25 at the side border 6 of the backing film 5. The temperature sensor 25 is calibrated directly in Celsius (Centigrade) with a 0.1° C. ensured accuracy (at 37° C.). The temperature sensor 25 is rated for a full 30° C. to 45° C. range with a power consumption of 0.5 mW-5 mW.

In the present embodiment, the integrated flexible printed electronic circuit 4 is a dynamic and flexible single-sided printed electronic circuit 4 on the lower absorbent printed circuit face 16 adapted to communicate with sensors 18 whether positioned above or below the electronic circuit 4 in the dressing system 1. However, in an alternative embodiment of the invention, the printed electronic circuit 4 can be a double-sided printed electronic circuit at the lower absorbent printed circuit face 16 and the back film face 17 of the secondary absorbent pad 2 to allow for more complex electronic circuits as required. In a further embodiment, the printed electronic circuit can be constructed from a stack of single- or double-sided printed absorbent pads. Technologies such as screen printing, inkjet printing, roll-to-roll printing, laser patterning or photolithographic etching may be utilised to manufacture the circuit and the at least one sensor.

In an alternative embodiment, the circuits and sensors and associated components are manufactured on a printed circuit board that may be detached for the dressing for ease of disposability.

The electronic circuit 4 and the sensors 18 are powered by a power source in the form of an integrated battery (not shown) connected to the flexible electronic circuit 4. A low profile battery having a voltage of up to 3 volts is suitable to power the dressing system 1 over a typical life cycle while batteries having a voltage of up to 6 volts can also be used. The battery is a low profile battery easily integrated into the dressing 1 having a high energy density and long cycle life whilst not suffering from a high self-discharge rate. In a preferred embodiment, the battery is a flat, flexible, environmentally-friendly battery.

The printed electronic circuit 4 includes a processor for processing data from the sensors 18, memory for storing data and programmes, a communications module for communicating the sensor data to a base station attended by a clinician, and an actuator for controlling actuation of the dressing system 1.

The communications module within the dressing 1 can connect to the base station by Bluetooth, WiFi, NFC, or other suitable wireless communications protocol. The dressing 1 transfers personal and private information to a data repository, which can be on a cloud server. A Raspberry pi (Trade Mark)/sensor node system can be used as a base station.

In the base of Bluetooth or WiFi transmission, the strength of the signal received at the base station may be monitored. This signal strength will vary according to the placement of the dressing and the orientation of the patient, e.g. the signal strength will decrease when the patient is lying on the bandage and integrated antenna. It therefore provides a means for monitoring the frequency of alteration of the position of the patient.

The wicking primary absorbent pad 2 is formed from a die-cut adhesive bandage material which allows for sufficient transfer of oxygen to a wound site while effectively preventing passage of microbes to the wound. A suitable material is a collagen biodegradable material integrated with a naturally occurring human growth factor protein with the human growth factor being released in an optimized topical delivery system. The wicking primary absorbent pad 2 rapidly absorbs exudates and interstitial fluids and optimises conditions for healing at the wound-dressing interface. Moreover, the primary absorbent pad 2 achieves a rapid capillary action response to quickly distribute absorbed exudate throughout the dressing 1 and create a sustained movement of exudate away from the wound bed. If desired, anti-bacterial silver nanoparticles can be incorporated into the primary absorbent pad 2 of the dressing 1 employing spinning techniques during manufacture or by coating the silver nanoparticles onto the primary absorbent pad 2 to aid in the prevention of infections and discourage the formation of biofilm development.

The secondary absorption pad 3 is formed from a flexible polymer such as a foam on which the electronic circuit 4 is printed while the backing film 5 is also formed from a polymer such as acrylic or polyurethane. The adhesive 15 on the backing film 5 is a silicone adhesive. Alternatively, an acrylic or other polymer-based adhesive 15 can be used. The release film 7 is formed from polyethylene terephthalate (PET).

The dressing system 1 can be manufactured in any suitable size as required in accordance with wound sizes. Typical dressing sizes are 10 cm×10 cm, 7.5 cm×7.5 cm, 20 cm×10 cm and 20 cm×20 cm.

The dressing system 1 is sterilised to kill microorganisms transferred during the manufacturing process. A suitable sterilization method is an ethylene oxide (EtO) sterilisation method which protects the electronic circuit 4 and sensors 18 from damage. This method of sterilisation is also preferred due to its handling ease, versatility and suitability for use with delicate medical dressings which could be damaged by other sterilisation methods such as heat sterilisation.

The battery powered sensor nodes have limited memory and can be deployed in difficult-to-access wound locations while the radio enables wireless communication to transfer data to the base station.

In an alternative embodiment of the invention, the electronic circuit 4 is printed directly onto the internal face of the backing film 5 and the sensors 18 are located in laser-cut cavities in the secondary absorption pad 3 for low profile sensor embedding. Accordingly, specific sensors 18 are integrated into the dressing system 1 without requiring relatively large secondary absorption pads 3 resulting in improved sensor readings. The secondary absorption pad 3 can then be directly fabricated onto the backing film 5.

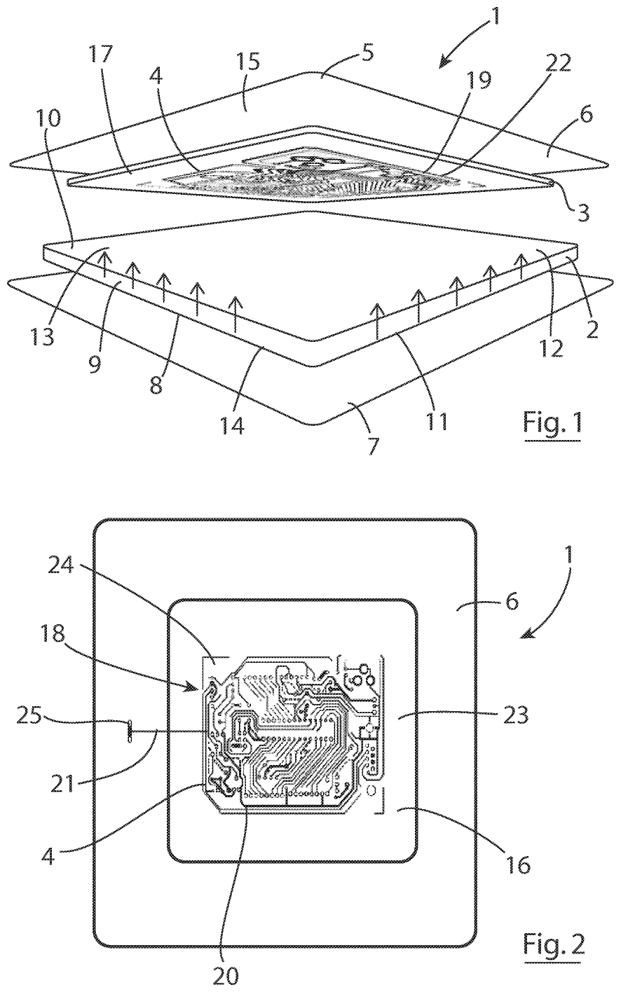

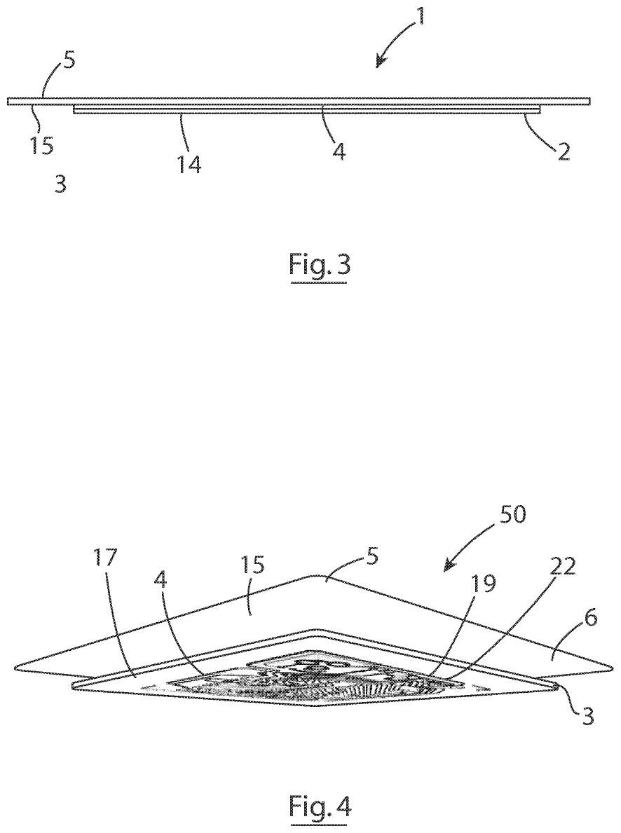

FIG. 4 illustrates another embodiment of an intelligent disposable wound dressing system of the invention indicated generally by the reference numeral 50 for applying the sensor and circuitry directly onto a wound. The same reference numerals as FIG. 1 are used for convenience. In this embodiment when a patient with a wound that presents themselves to a doctor or a clinician with an open weeping wound the dressing system can be placed directly on the wound without any absorbent pad. It will be appreciated that in one embodiment the electronic circuit can be printed on the adhesive film backing 5 with no need for the secondary pad 3 as shown in FIG. 4. The printed sensor and electronic layer can be laid directly on the wound and important information about the wound in real time can be communicated to the clinician.

In use, data from the sensors 18 is harvested and processed for optimal wound monitoring and healing. Various data processing methods can be employed. A data driven approach is preferred to reduce the amount of sampled data by keeping the sensing accuracy within an acceptable level for the dressing system 1. The sampled data has a strong spatial and/or temporal correlation so there is no need to communicate redundant information.

A data generation method can be used to construct a model describing the sensed parameters so that queries can be answered using the model instead of the actual sensed data. Two instances of a communication model can be provided—one residing at the sink and the other at source sensors 18. The model at the sink can be used to answer queries without requiring any communication thus reducing energy consumption while the sensors 18 sample the data.

As indicated above, the software aspect of the invention is developed through a Trade Mark system and programmed using a suitable language such as Java, Objective C and Java Script (Trade Mark). An algorithm gathers all sensor 18 data and then generates wound healing conclusions based on the gathered data.

In one embodiment generated data can also be transferred to a storage solution for off-site archiving using Amazon (Trade Mark) Simple Queue Service (SQS) from remote dressing systems 1 or base stations. SQS is preferred due to its reliability, scalability and security. Amazon (Trade Mark) Web Services can be used to transfer the data.

ASP.NET can be used for creating a web application of the software platform which can be developed using the Java (Trade Mark) language. ASP.NET enables real-time bi-directional communication between client and server and can be used to support clients on Android, iPhone (Trade Mark) and C# clients like Windows Phone and Windows 8 (Trade Mark).

HTML5 can be used to create an app and website that functions like a desktop application allowing simultaneous access to all users. More particularly, data can be transferred to the downloaded app on a handheld device to provide clinicians with continuous data for monitoring and analysis of wounds. The app enables real-time bi-directional communication between client and server.

The HTML5 created app functions even when not connected and when the system is offline. The offline feature enables storage of data in a cache or in such a way that allows the data to be retained even if the relevant page is reloaded.

The dressing system 1 of the invention is provided with security features while data from the dressing 1 is encrypted and clinicians require a password to access data.

Health Information Privacy (HIPAA) regulations are also employed to protect data stored on servers.

As indicated above, the generated data of the sensors 18 is compared to a prediction model algorithm embedded within a Trade Mark computer system and if a sensed value falls within an application-dependent tolerance, then the model is considered valid.

Time series forecasting is the preferred data prediction technique used with the dressing. A set of chronological values obtained by periodic samplings is used to predict a future value in the same series. To build a large data bank with statistically relevant data, a large patient number with the same wound type (e.g. patients suffering from an arterial ulcer) is employed to generate a strong profile. The set of chronological values obtained in this manner explicitly considers the internal structure of the harvested data.

Each patient's medical history is employed to create a profile within the software (e.g. diabetic, High BMI, Low BMI). This information is useful to identify similarities in patient wound healing and predict outcomes dependent on different medical backgrounds. The algorithm is based on current practices and compared to the data generated. The data generated across the patient studies is used to identify the stages of wound healing.

As an initial starting point, the wound healing stages are based on monitoring wound appearance and wound area. These methodologies are compared to the sensor data recorded and comparisons are made. Patient profiles are generated indicating similarities in wound type and condition and are compared and inputted into a wound specific database.

The data generated from the dressing system 1 is compared to that of similar patient types and data generated. Firstly, t-test analyses are undertaken. T-test statistical significance will indicate whether or not the difference between two groups' averages most likely reflect a “real” difference in the population from which the groups were sampled.

To test the accuracy of the methodology, it is significant to compare the profile against a validation set. The validation set is made up of patients whose results are already known but the data has not been used to provide the original profile. The validation set is divided into a training set and a test set.

Biostatistical analysis (sRoc) may be carried out using a test/train methodology. A calculation is performed of the highest AUC (0.999) and lowest P-value (0.012) for the parameter measured. This methodology identifies the most confident data to use when determining predictive trends of wound healing.

CART analysis compares the considered parameter (e.g. moisture/impedance senso readings) with outcome (healing time). A threshold determines whether or not the parameter is above (slow healing) or below (fast healing) the threshold. The optimal parameter threshold associated with wound healing is therefore identified in this manner to predict trends in wound healing.

In the context of the present invention the term “Integrated” is used to mean a single dressing system embodied as a single product, as hereinbefore described with reference to the description and/or figures.

The embodiments in the invention described with reference to the drawings comprise a computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. CD ROM, or magnetic recording medium, e.g. a memory stick or hard disk. The carrier may be an electrical or optical signal which may be transmitted via an electrical or an optical cable or by radio or other means.

In the specification the terms “comprise, comprises, comprised and comprising” or any variation thereof and the terms include, includes, included and including” or any variation thereof are considered to be totally interchangeable and they should all be afforded the widest possible interpretation and vice versa.

The invention is not limited to the embodiments hereinbefore described but may be varied in both construction and detail.

1. An integrated dressing system for a wound comprising:
an absorbent pad;
at least one sensor for detecting wound data or dressing data, wherein the at least one sensor includes a moisture sensor;
a flexible electronic circuit communicable with the at least one sensor;
a backing film adapted to cooperate with the absorbent pad, wherein the flexible electronic circuit and the at least one sensor are printed directly on the backing film; and
a communications module adapted to communicate the wound data or dressing data from the flexible electronic circuit to a processing device.

2. The dressing system of claim 1, wherein the dressing system is a disposable one time use dressing system.

3. The dressing system of claim 1, wherein the communications module comprises a wireless communications module.

4. The dressing system of claim 1, wherein the at least one sensor comprises an internal sensor in the absorbent pad.

5. (canceled)

6. The dressing system of claim 1, wherein the moisture sensor comprises an impedance sensor, a capacitance sensor, a resistance sensor or a strain sensor.

7-9. (canceled)

10. The dressing system of claim 1, wherein the at least one sensor comprises an internal sensor integrated within the absorbent pad.

11. (canceled)

12. The dressing system of claim 1, wherein the at least one sensor is positioned on a border of the dressing.

13. The dressing system of claim 12, wherein the at least one sensor comprises a temperature sensor positioned on a peripheral region to the wound.

14. The dressing system of claim 1, wherein the at least one sensor comprises a sensor network.

15. (canceled)

16. The dressing system of claim 1, further comprising a power source configured to power the flexible electronic circuit.

17-18. (canceled)

19. The dressing system of claim 1, wherein the backing film comprises a flexible printed circuit board material.

20. The dressing system of claim 1, wherein the backing film comprises a polyurethane backing film.

21. The dressing system of claim 1, wherein the flexible electronic circuit is printed on a single side of the absorbent pad.

22. The dressing system of claim 1, wherein the flexible electronic circuit is printed on two sides of the absorbent pad.

23. The dressing system of claim 1, wherein the absorbent pad comprises a secondary absorption pad in the dressing.

24. (canceled)

25. The dressing system of claim 1, wherein the flexible electronic circuit comprises:
a processor configured to process data provided by the at least one sensor; and
a memory device configured to store the wound data.

26. The dressing system of claim 1, wherein the processing device comprises a base station configured to receive the wound data communicated from the communications module.

27-28. (canceled)

29. A method for monitoring a wound, comprising:
applying a dressing system having at least one printed moisture sensor to a peripheral region of the wound;
detecting wound or dressing data at the sensor;
communicating the detected wound data via a printed flexible electronic circuit in the dressing system to a base station; and
displaying the data at the base station.

30. The method of claim 29, wherein the detected wound data is wirelessly communicated to the base station, wherein a biostatistical analysis is performed on the processed data to identify a wound healing predictive trend.

31. The method of claim 29, wherein the wound data comprises pH data, temperature data, moisture data, pressure data or bacteria data.

32-37. (canceled)
